(19) United States
(12) Patent Application Publication
(10) Pub. No.: US 2016/0231396 A1
(43) Pub. Date: Aug. 11, 2016

(54) MAGNETIC RESONANCE IMAGING APPARATUS AND IMAGING PARAMETER SETTING ASSISTING METHOD

(71) Applicant: HITACHI MEDICAL CORPORATION, Tokyo (JP)

(72) Inventors: Kentaro SUNAGA, Tokyo (JP); Yutaka YOSHIDA, Tokyo (JP)

(73) Assignee: Hitachi Medical Corporation, Tokyo (JP)

(21) Appl. No.: 15/023,535

(22) PCT Filed: Oct. 2, 2014
(86) PCT No.: PCT/JP2014/076350

§ 371 Date: Mar. 21, 2016

(30) Foreign Application Priority Data
Oct. 16, 2013 (JP) 2013-215352

(51) Int. Cl. G01R 33/28; G01R 33/54; A61B 5/055

(52) U.S. Cl. CPC G01R 33/288; A61B 5/055; G01R 33/543

(57) ABSTRACT

In order to provide an MRI apparatus that can efficiently approximate a specific absorption rate or a magnetic field variation rate per unit time of a magnetic flux density to a target value, the present invention is characterized in that suggestions of imaging parameters related to a control subject and change directions of the imaging parameters when the control subject is input based on an input operation and that the control unit further calculates values of the selected control subjects based on the changed imaging parameter values.

### TECHNICAL FIELD

The present invention relates to a magnetic resonance imaging (hereinafter, referred to as “MRI”) apparatus.

### BACKGROUND ART

An MRI apparatus two-dimensionally or three-dimensionally measures Nuclear Magnetic Resonance (NMR) signals generated by an atomic nucleus spin that comprises tissues of an object, particularly a human body to image forms and functions of the head, abdomen, extremities, and the like. During imaging, the NMR signals are provided with phase encodings different depending on the gradient magnetic field, frequency-encoded, and measured as time-series data. The measured NMR signals are two-dimensionally or three-dimensionally Fourier-transformed in order to reconstruct an image.

Normally, a plurality of pulse sequences are used for an object in a series of imaging operations, and high-frequency magnetic field pulses (hereinafter, referred to as “RF pulse”) are continuously irradiated. Therefore, an MRI apparatus can store pulse sequence combinations and an irradiation order to be used for a series of irradiations. Such pulse sequence combinations are referred to as protocols.

Although reconstruction image quality, an imaging time, and a Specific Absorption Rate (SAR) in magnetic resonance imaging are greatly different depending on the pulse sequence to be used for imaging, great differences are caused by differences of the imaging parameter setting values (a field of view (FOV), a pulse repetition time (TR), an echo time (TE), an inversion time (TI), a slice thickness, the number of slices, the number of imaging matrices, the number of signal additions, and the like) even in case of a similar pulse sequence.

Therefore, in consideration of a burden on an object, an operator of an MRI apparatus is required to meticulously set imaging parameter values by considering, for example, physical conditions, an imaging time, a disease type, a diagnosis site, and imaging region in order to acquire an image that allows a doctor to perform an accurate diagnosis.

In the non-patent literature 1, an apparatus state is divided into three stages, and they are specified as a normal operation mode, a first level controlled operating mode, and a second level controlled operating mode starting from a lower SAR according to a time average SAR value while a gradient magnetic field is being applied. In order to reduce a burden on an object, an object generally has to be imaged in the normal operation mode or the first level controlled operating mode, and a pregnant object and an object having difficulty adjusting the body temperature have to be imaged only in the normal operation mode.

There are various interrelationships between each imaging parameter and a SAR. For example, when a pulse repetition time (TR) is shortened, a time-average SAR is increased. An operator is required to search and set an optimal imaging parameter value in an allowable operation mode under restriction based on such an interrelationship.

As shown in the non-patent literature 1, it is required to perform imaging when an apparatus state is a normal operation mode or a first level controlled operating mode in order to reduce a burden on an object. Therefore, if a SAR in each pulse sequence or a magnetic field variation rate (dB/dt) per unit time t of a magnetic flux density B exceeds an allowable value in an operation mode capable of imaging, imaging parameters need to be adjusted in order to reduce these rates.

Although there is no problem if imaging parameters were adjusted for a stored protocol so that SAR and dB/dt values are within those allowed in an operation mode capable of imaging in a pulse sequence, a SAR particularly varies depending on the physical characteristics (height, weight, and the like) of an object, and there are some cases where the SAR of the stored protocol is a value outside a range of the operation mode capable of imaging.

Also, in an examination using a contrast agent, it is necessary to reduce a SAR in each pulse sequence because there are restrictions where a pulse sequence order cannot be changed, where a waiting time cannot be added between pulse sequences, and the like. Additionally, in an examination for examining a plurality of sites such as a contrast examination for lower extremities, it is required to adjust imaging parameters to set conditions capable of imaging so as not to change the contrast of an image to be acquired in each pulse sequence. Therefore, it is required to adjust imaging parameters in a pulse sequence so as not to exceed SAR and dB/dt allowable values and so as to maintain image quality required for the examination.

The patent literature 1 discloses an MRI apparatus having a display unit that displays setting values of imaging parameters interrelated with each other and the settable ranges (variable range) and additionally changes and re-displays the settable ranges (variable range) of the other related imaging parameter values according to a change of an imaging parameter value.

### CITATION LIST

### Patent Literature

PTL 1: Unexamined Patent Publication No. 6-90926

### Non-Patent Literature

NPTL 1: “IEC 60601-2-33 Ed. 3: Medical electrical equipment—Part 2-33: Particular requirements for the basic safety and essential performance of magnetic resonance equipment for medical diagnosis” by International Electrotechnical Commission

### SUMMARY OF INVENTION

### Technical Problem

Although the purpose of the technique described in the patent literature 1 is to suggest specific values of imaging parameters at which the imaging parameters become conditions capable of imaging for one pulse sequence, an operator is required to repeatedly check whether or not the imaging parameters become conditions capable of imaging by setting specific parameters in order to reduce a SAR and a dB/dt.

Therefore, the purpose of the present invention is to provide an MRI apparatus that can adjust imaging parameters more efficiently.

### Solution to Problem

The magnetic resonance imaging apparatus related to the present invention is comprised of a static magnetic field generation source that generates a static magnetic field in a space accommodating an object, a gradient magnetic field generating unit that generates a gradient magnetic field to be superimposed on the static magnetic field, a high-frequency magnetic field generating unit for irradiating a high-frequency magnetic field pulse to the object, a signal detection unit that detects a nuclear magnetic resonance signal to be generated from the object, a sequencer that controls the static magnetic field generation source; the gradient magnetic field generating unit; the high-frequency magnetic field generating unit; and the signal detection unit according to a pulse sequence, and a control unit that has a storage device; an input device; an output device; and a CPU, when a control subject is input based on an input operation, the control unit displays suggestions of imaging parameters related to the control subject and change directions of the imaging parameters, and when the displayed imaging parameters are changed, the control unit further calculates values of the selected control subjects based on the changed imaging parameter values.

### Advantageous Effects of Invention

According to the present invention, an MRI apparatus that can adjust imaging parameters more efficiently can be obtained.

### BRIEF DESCRIPTION OF DRAWINGS

FIG. 1 is a view for explaining the overall configuration of the MRI apparatus that is an embodiment of the present invention.

FIG. 2 is a view for explaining the procedure before starting imaging in the embodiment described in FIG. 1.

FIG. 3 is a view for explaining the procedure to adjust parameters in the embodiment described in FIG. 2.

FIG. 4 is a view for explaining an example of a parameter display window.

FIG. 5 is a view for explaining arithmetic expression data that is a database example.

FIG. 6 is a view for explaining the procedure to change parameters.

FIG. 7 is a view for explaining an example of a parameter change screen of a SAR.

FIG. 8 is a view for explaining an example of a parameter change screen of a dB/dt.

FIG. 9 is a view for explaining the procedure to re-adjust parameters.

FIG. 10 is a view for explaining an example of a parameter change screen.

FIG. 11 is a view for explaining an example of a parameter change screen after changing parameters.

FIG. 12 is a view for explaining an example of a parameter change screen after changing a target value.

FIG. 13 is a view for explaining another embodiment of the parameter change screen described in FIG. 10.

FIG. 14 is a view for explaining the other embodiment of the parameter change screen described in FIG. 10.

FIG. 15 is a view for explaining an example of a parameter change screen of a method for selecting a parameter arbitrarily.

FIG. 16 is a view for explaining operations of the method described in FIG. 15.

FIG. 17 is a view for explaining an example of a parameter change screen in case of specifying a change plan.

FIG. 18 is a view for explaining the parameter adjustment procedure in a case where a plurality of pulse sequences are selected.

FIG. 19 is a view for explaining an example of a parameter change screen in a case where a plurality of pulse sequences are selected.

FIG. 20 is a view for explaining an example of a parameter change screen after changing parameters in a case where a plurality of pulse sequences are selected.

### DESCRIPTION OF EMBODIMENTS

Hereinafter, an embodiment of the MRI apparatus related to the present invention (hereinafter, described as an embodiment) will be described in detail using the attached diagrams. Additionally, in the description of embodiments of the present invention, the same numerals are provided for the elements and procedures with the same functions in the diagrams, and the repeated descriptions will be omitted. In the present description, “calculation” includes not only four basic arithmetic operations by an algebra equation but also processes such as acquiring a desired value by retrieving data generated based on results experimented in advance, simulation results, calculation results, and the like.

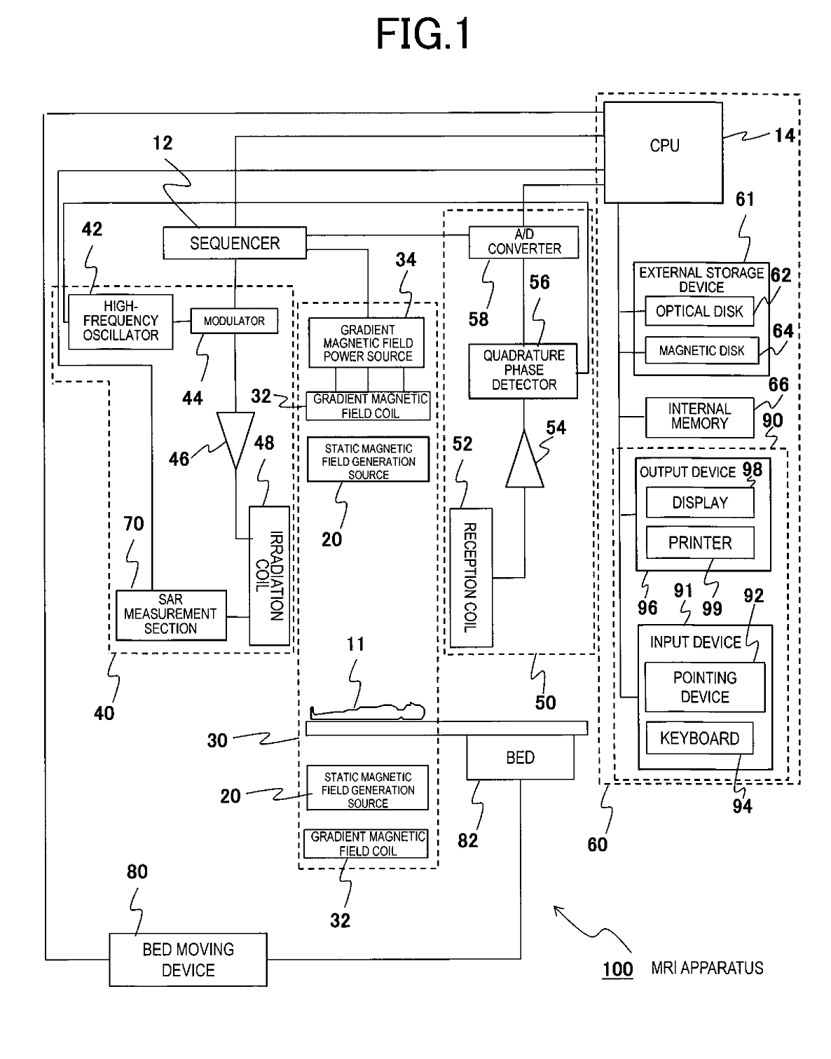

First of all, the overview of the MRI apparatus of an embodiment will be described based on FIG. 1. An MRI apparatus 100 is an apparatus that acquires a tomographic image of an object 11 by utilizing the NMR phenomenon. As shown in FIG. 1, the MRI apparatus 100 is comprised of a static magnetic field generation source 20, a gradient magnetic field generating unit 30, a sequencer 12, a high-frequency irradiation unit 40, a signal detection unit 50, and a control unit 60.

The static magnetic field generation source 20 generates a homogeneous static magnetic field in a direction orthogonal to the body axis of the object 11 in case of a vertical magnetic field method and in the body-axis direction of the object 11 in case of a horizontal magnetic field method in a static magnetic field space accommodating the object 11. The static magnetic field generation source 20 of a permanent magnet method, normal conducting method, or superconducting method is disposed around the object 11.

The gradient magnetic field generating unit 30 has a gradient magnetic field coil 32 that generates gradient magnetic fields in the three axis directions X, Y, and Z that are a coordinate of the MRI apparatus 100 (stationary coordinate system) by superimposing them on the static magnetic field space and a gradient magnetic field power source 34 that drives the respective gradient magnetic field coils. By driving the gradient magnetic field power source 34 of the respective coils according to the command, i.e. control from the sequencer 12 to be described later, gradient magnetic fields Gx, Gy, and Gz are generated in the three axis directions X, Y, and Z.

In imaging, a slice plane (imaging cross section) for the object 11 by applying a slice direction gradient magnetic field pulse (Gₛ) in a direction orthogonal to the slice plane, a phase encoding direction gradient magnetic field pulse (Gₚ) and a frequency encoding direction gradient magnetic field pulse (G_{f}) are applied in the other two directions orthogonal to the slice plane and to each other, and then positional information of the respective direction is encoded to echo signals.

The sequencer 12 repeatedly applies a high-frequency magnetic field pulse (RF pulse) and a gradient magnetic field pulse in a predetermined pulse sequence. The sequencer 12 operates based on the control of a central processing unit (hereinafter, described as CPU) 14 and transmits various commands, i.e. control required to collect tomographic image data of the object 11 to the gradient magnetic field generating unit 30, the high-frequency irradiation unit 40, and the signal detection unit 50.

The high-frequency irradiation unit 40 irradiates an RF pulse to the object 11 in order to cause nuclear magnetic resonance to atomic nucleus spins of atoms composing living tissue of the object 11. The high-frequency irradiation unit 40 includes a high-frequency oscillator 42, a modulator 44, a high-frequency amplifier 46, and an irradiation coil 48 that is a high-frequency coil on the transmission side. An RF pulse output from the high-frequency oscillator 42 is amplitude-modulated by the modulator 44 at a timing by a command from the sequencer 12, the amplitude-modulated RF pulse is amplified using the high-frequency amplifier 46 and supplied to the irradiation coil 48 disposed in the vicinity of the object 11, and then an electromagnetic wave is irradiated to the object 11.

The signal detection unit 50 detects an echo signal that is an NMR signal to be emitted by nuclear magnetic resonance of atomic nucleus spins composing living tissue of the object 11. The signal detection unit 50 includes a reception coil 52 that is a high-frequency coil on the reception side, a signal amplifier 54, a quadrature phase detector 56, and an analog/digital converter (hereinafter, described as A/D converter) 58. A response NMR signal excited in the object 11 by electromagnetic waves irradiated from the irradiation coil 48 is detected by the reception coil 52 disposed in the vicinity of the object 11, is amplified by the signal amplifier 54, and is divided into two orthogonal system signals by the quadrature phase detector 56 at a timing by a command from the sequencer 12, and then the respective system signals are converted into digital amounts by the A/D converter 58 and are sent to the control unit 60.

The control unit 60 processes various data and displays and stores the process results. The control unit 60 includes a processor such as the CPU 14, a storage device such as an internal memory 66, an external storage device 61 such as an optical disk 62 and a magnetic disk 64, and an input/output device 90. When the signal detection unit 50 receives signals and data, the CPU 14 executes processes such as signal processing and image reconstruction using the internal memory 66 as a work area, displays a tomographic image of the object 11 that is the process result on an output device 96, and stores the image in the external storage device 61 (such as the magnetic disk 64).

The input/output device 90 inputs and outputs various control information of the MRI apparatus 100 and control information to be processed by the control unit 60, or specifically receives inputs of and displays imaging parameters of a pulse sequence and the like. The input/output device 90 is comprised of, for example, a pointing device 92 such as a trackball; a mouse; a pad; and a touch panel, input devices 91 including a keyboard 94, a display 98 such as a cathode-ray tube (hereinafter, described as CRT) and a liquid crystal display (hereinafter, described as LCD), and the output device 96 including a printer 99.

The input devices 91 may be arranged in the vicinity of the output device 96 to control the MRI apparatus 100 interactively by providing a command to execute various processes through the pointing device 92 while an operator checks the display 98, for example. Also, it may be configured so as to perform inputs by disposing a touch panel operating as the input device 91 on the display surface of the display 98 and selecting or operating displayed contents on the display 98.

Additionally, in FIG. 1, the object 11 is placed on the top plate of a bed 82 and accommodated by a bed moving device 80 in a static magnetic field space that is an imaging space. The irradiation coil 48 and the gradient magnetic field coil 32 on the transmission side are installed in the static magnetic field space where the object 11 is accommodated, opposite to the object 11 in case of the vertical magnetic field method or so as to surround the object 11 in case of the horizontal magnetic field method. Also, the reception coil 52 on the reception side is installed opposite to the object 11 or so as to surround the object 11.

A clinically prevalent nuclide to be imaged by the MRI apparatus 100 is currently the hydrogen atomic nucleus (proton) that is a main component of the object 11. By imaging information about spatial distribution of the proton density and that of a relaxation time in an excited state, forms and functions of the head, abdomen, extremities, and the like are imaged two- or three-dimensionally.

Hereinafter, the MRI apparatus and the imaging parameter setting assisting method of the present invention will be described. In the present invention, at least one of a specific absorption rate and a magnetic field variation rate per unit time of a magnetic flux density is set as a control subject, a suggestion of imaging parameters related to the control subject and a suggestion of change directions of the imaging parameters are displayed when the control subject is input based on an input operation, and then a value of the selected control subject is calculated based on a changed imaging parameter value when a displayed imaging parameter is changed.

For example, when a control subject input based on an input operation is a specific absorption rate, a suggestion of imaging parameters related to the specific absorption rate is displayed, and information about a suggestion of change directions of the imaging parameters is displayed. Alternatively, when a control subject input based on an input operation is a magnetic field variation rate per unit time of a magnetic flux density, a suggestion of parameters related to the magnetic field variation rate per unit time is displayed, and information about a suggestion of change directions of the imaging parameters for reducing the magnetic field variation rate is further displayed.

Hereinafter, the respective embodiments of the present invention will be described.

### First Embodiment

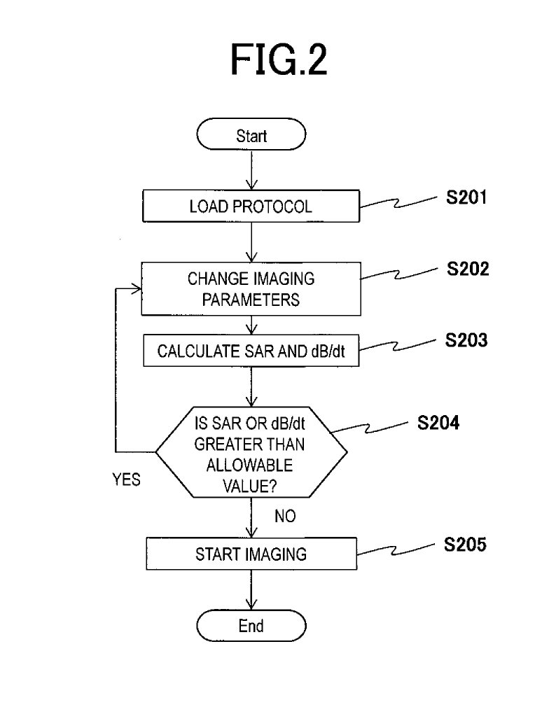

The first embodiment 1 of the present invention will be described. First of all, the flow before starting imaging using the MRI apparatus 100 with the above configuration will be described with FIG. 2. For the object 11 to be examined, imaging can be started because imaging conditions are satisfied in a case where values of imaging indexes (a SAR and a dB/dt) are within allowable values in an operation mode capable of imaging when an RF pulse that is an electromagnetic wave is irradiated according to the pulse sequence stored in the internal memory 66 and the like of the MRI apparatus 100.

First, a protocol that is a pair of pulse sequences is loaded to the MRI apparatus 100 (Step S201). Specifically, the CPU 14 loads a protocol corresponding to predetermined conditions (for example, living body information such as a height, weight, and the like of the object 11) to the internal memory 66. The protocol may be stored in the external storage device 61 in advance or may be input by an operator from the input device 91 to generate the protocol.

Following the above, imaging parameter adjustment is performed for a pulse sequence in a protocol (Step S202). When an operator selects an arbitrary pulse sequence using the input device 91 to change the imaging parameter values, the CPU 14 identifies a pulse sequence to be changed and obtain change values of the imaging parameters.

Following the above, a SAR and a dB/dt are calculated using the change values of the imaging parameters (Step S203). The CPU 14 identifies an arithmetic expression and an imaging parameter to be used, performs calculation, and then outputs the calculation result. Then, the CPU 14 determines whether or not the calculated values of imaging indexes is greater than an allowable value in an operation mode capable of imaging (Step S204).

In a case where a SAR or dB/dt value is greater than an allowable value (Step S204: Yes), the CPU 14 provides a correcting command such as displaying a window notifying an imaging index exceeds the allowable value on the display 98 to an operator, and then goes back to Step S202 to change an imaging parameter. In a case where SAR and dB/dt values are less than an allowable value (Step S204: No), the CPU 14 can execute a pulse sequence in which the pulse sequence was changed and controls so as to start imaging (Step S205).

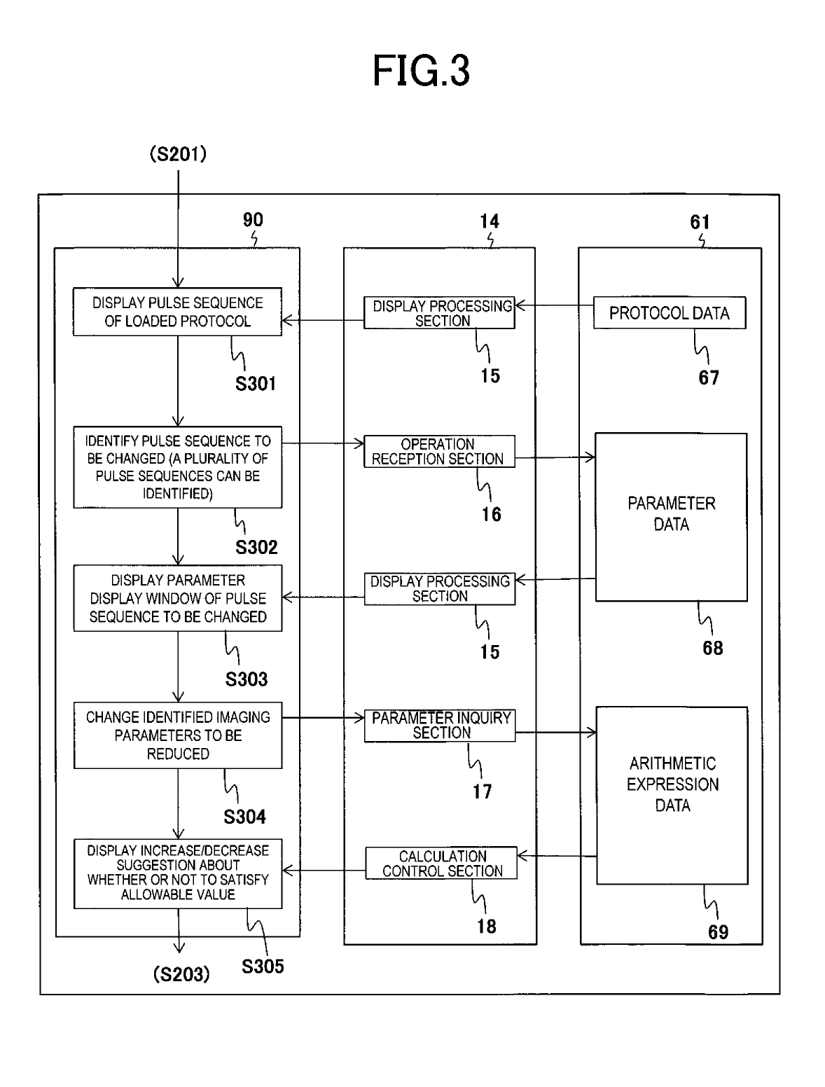

Here, the processes of the control unit 60 to optimize the Step S202 process will be described. FIG. 3 is a functional block diagram showing processing contents of the control unit 60, and specifically is a diagram showing a flow of the processes to be executed through the input/output device 90 that has the input device 91 and the output device 96, the types of processes to be executed by the CPU 14, and the relationship with data to be stored in the external storage device 61 such as a database generated in the magnetic disk 64 (including a case of temporary storage in the internal memory 66).

Additionally, the CPU 14 functions as a display processing section 15 that displays various data and the like on the output device 96, an operation reception section 16 that receives operations from the input device 91, a parameter inquiry section 17 that obtains change values of imaging parameters and checks whether or not an allowable value of an imaging index is satisfied, and a calculation control section 18 that calculates the imaging index and outputs the calculation result according to a processing purpose.

Through Step S201 described in FIG. 2, the control unit 60 displays a pulse sequence included in a loaded protocol on the input/output device 90 (Step S301). Because protocol data 67 extracted from a database generated in the external storage device 61 has been loaded in the internal memory 66, the display processing section 15 of the CPU 14 displays a plurality of pulse sequences included in the protocol data 67 in a format such as a table on the output device 96 so that an operator can select a pulse sequence. Additionally, the protocol data 67 is data identifiably stored in the external storage device 61 and the like by providing identification information each time a pulse sequence or the pair is generated and can be extracted by retrieving based on the identification information.

Next, a pulse sequence to change imaging parameters is identified (Step S302). When an operator selects a pulse sequence a pulse sequence to change imaging parameters from among a plurality of pulse sequences displayed on the output device 96 using the input device 91, the operation reception section 16 of the CPU 14 receives identification information of the pulse sequence from the input device 91 to identify a pulse sequence to be changed. The identification information is also set as conditions to extract parameter data 68 of the pulse sequence from the external storage device 61. Additionally, a plurality of pulse sequences to be changed may be identified.

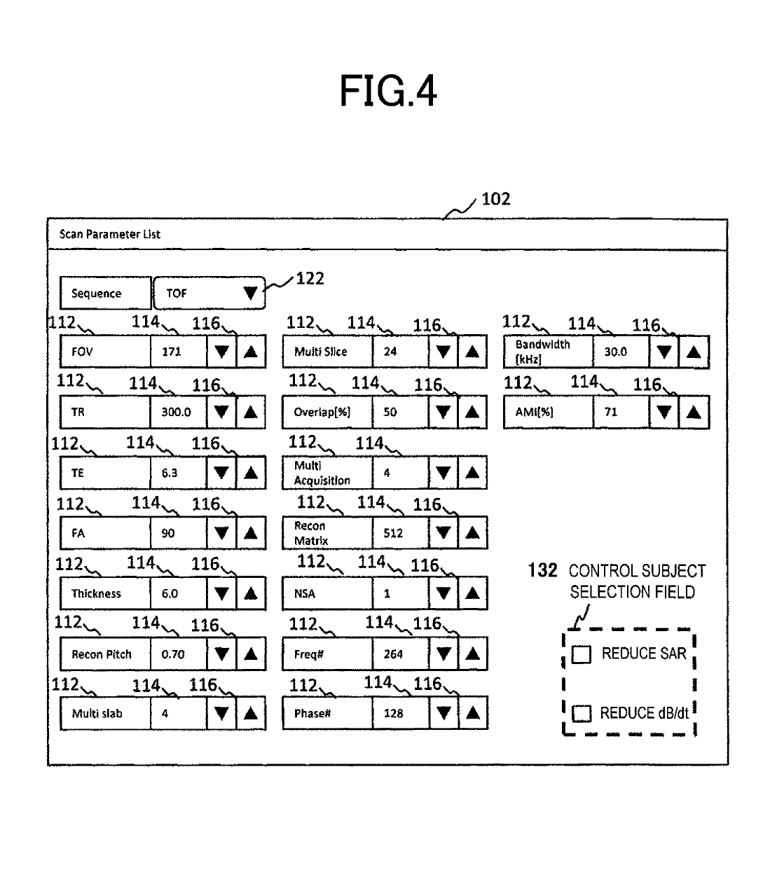

Next, the imaging parameters of the pulse sequence to be changed are displayed (Step S303). The display processing section 15 of the CPU 14 sets the parameter data 68 extracted from the external storage device 61 for the corresponding items on a parameter display window shown in FIG. 4 and displays them on the output device 96. Additionally, the parameter data 68 is imaging parameters comprising a plurality of property information such as imaging conditions of the pulse sequence as shown in FIG. 4, may be data associated with the identification information of the pulse sequence, and may be data subordinate to the pulse sequence.

As an example of a parameter display window 102 displayed on the display 98, item fields 112 of the respective parameters, setting value fields 114 that displays current setting values of the respective item parameters, operation fields 116 for operations to increase/decrease or change parameter values, a control subject selection field 132 that specifies whether or not either or both of a SAR and a dB/dt to be reduced, a sequence specifying field 122 for specifying a pulse sequence to be changed from a plurality of pulse sequences, and the like are arranged so as to be listed and displayed as shown in FIG. 4.

Next, a reduction target is specified to obtain change values of imaging parameters (Step S304). When an operator selects a SAR or dB/dt as the reduction target using the input device 91, the parameter inquiry section 17 of the CPU 14 identifies the reduction target and temporarily stores it in the internal memory 66. Furthermore, when an operator changes the setting value fields 114 of imaging parameters displayed on the parameter display window 102 through the input device 91 by operating the operation fields 116, the parameter inquiry section 17 of the CPU 14 obtains changed setting values and temporarily stores them in the internal memory 66. Additionally, by identifying a reduction target, imaging parameters, a function that is an arithmetic expression, and the like to be used are identified by arithmetic expression data 69.

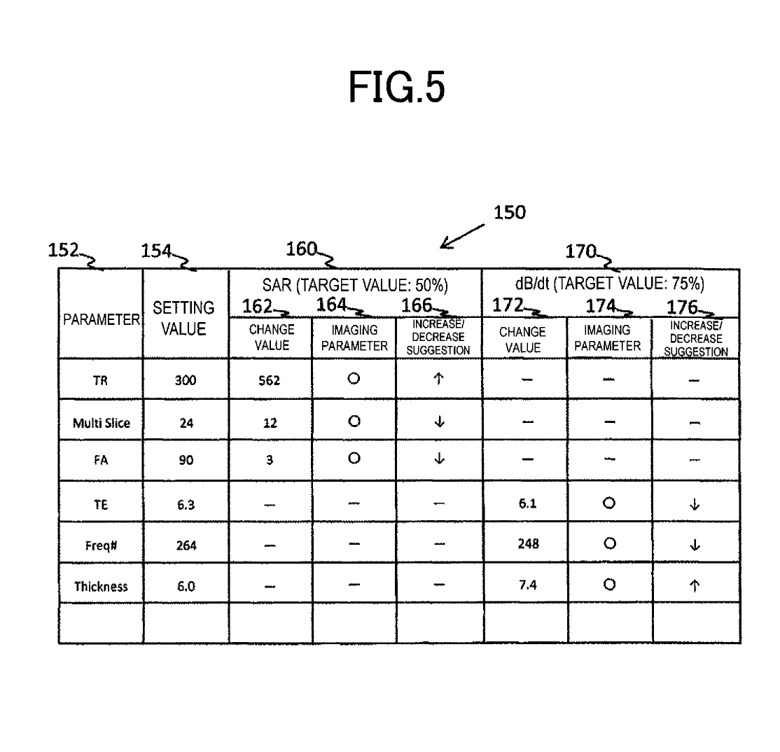

The arithmetic expression data 69 is data showing which imaging parameters (variables) and what type of arithmetic expression (not shown in the diagram) are used respectively in SAR and dB/dt calculations as shown in FIG. 5. The data also includes data such as information about current values of the respective variables, change values for approximating a SAR and a dB/dt to target values, and increase/decrease suggestions. A region (field) to store each data is provided for a single record when generated as a table in a database.

Next, whether or not the changed imaging parameters satisfy allowable values is determined, and then increase/decrease suggestions are displayed for each parameter in case of not satisfying the allowable values (Step S305). The calculation control section 18 of the CPU 14 included in the control unit 60 calculates SAR and dB/dt values using changed values temporarily stored in the internal memory 66 and determines whether or not to satisfy the allowable values respectively. Then, in case of not satisfying the allowable values, this is notified to an operator, and whether or not to increase or decrease each parameter is suggested by a means such as highlighting each parameter. In case of satisfying the allowable values, the procedure proceeds to Step S203 in order to calculate a SAR and a dB/dt actually.

FIG. 5 shows an example of data stored in the internal memory 66 based on the database stored in the external storage device 61. A database 150 shown in FIG. 5 includes parameter fields 152 where the respective parameters are stored and setting value fields 154 where setting values for the respective parameters are stored. The database 150 further includes a SAR target value field 160 for storing a set target value of a SAR that is a control subject, SAR change value fields 162 for storing change values of parameters related to SAR target value calculation, imaging parameter fields 164 for storing whether or not the parameters are related to SAR target calculation, and increase/decrease suggestion fields 166 that show whether to increase or decrease setting values in order to achieve SAR targets.

Additionally, for dB/dt that is a control subject, the database 150 includes a dB/dt target value field 170 for storing dB/dt target values, dB/dt change value fields 172 for storing change values of parameters related to dB/dt target value calculation, imaging parameter fields 174 for storing whether or not the parameters are related to dB/dt target calculation, and increase/decrease suggestion fields 176 that show whether to increase or decrease setting values in order to achieve dB/dt targets.

The increase/decrease suggestion fields 166 of SAR and the increase/decrease suggestions fields 176 of dB/dt are determined based on an arithmetic expression for calculating a SAR or dB/dt that is a control subject. When the target values are relaxed, the increase/decrease directions of the increase/decrease suggestion fields 166 and the increase/decrease suggestion fields 176 are reversed. When one of a case of tightening the target values and a case of relaxing the target values is stored, an increase/decrease direction can be determined by reversing the increase/decrease direction in the opposite case a mentioned above.

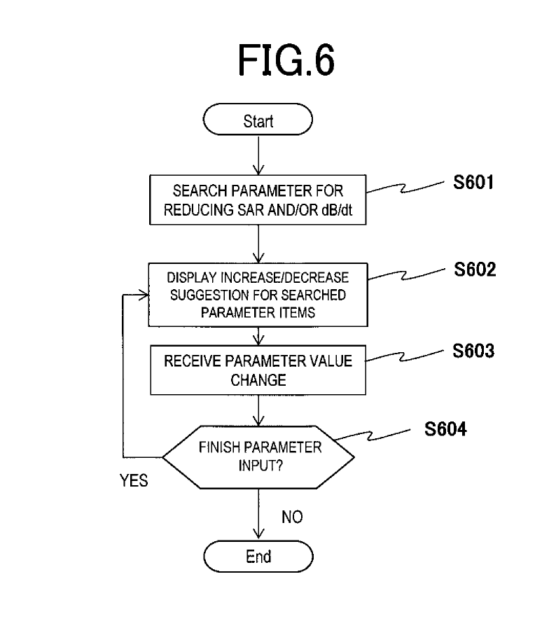

Furthermore, a specific example of Step S304 will be described using FIG. 6. When the CPU 14 of the control unit 60 identifies a reduction target, a search is performed for imaging parameters to decrease a SAR and a dB/dt or either of them as needed as shown in FIG. 6 (Step S601). The parameter inquiry section 17 identifies imaging parameters to be used in for SAR and dB/dt calculations from imaging parameters of the database 150 exemplified in arithmetic expression data shown in FIG. 5.

Next, suggestions to increase or decrease the searched imaging parameters are displayed based on the data of the database exemplified in FIG. 5 (Step S602). The parameter inquiry section 17 obtains current values for the imaging parameters and calculates SAR and dB/dt values or either of them as needed using the current values in order to determine increase/decrease suggestions for the current values.

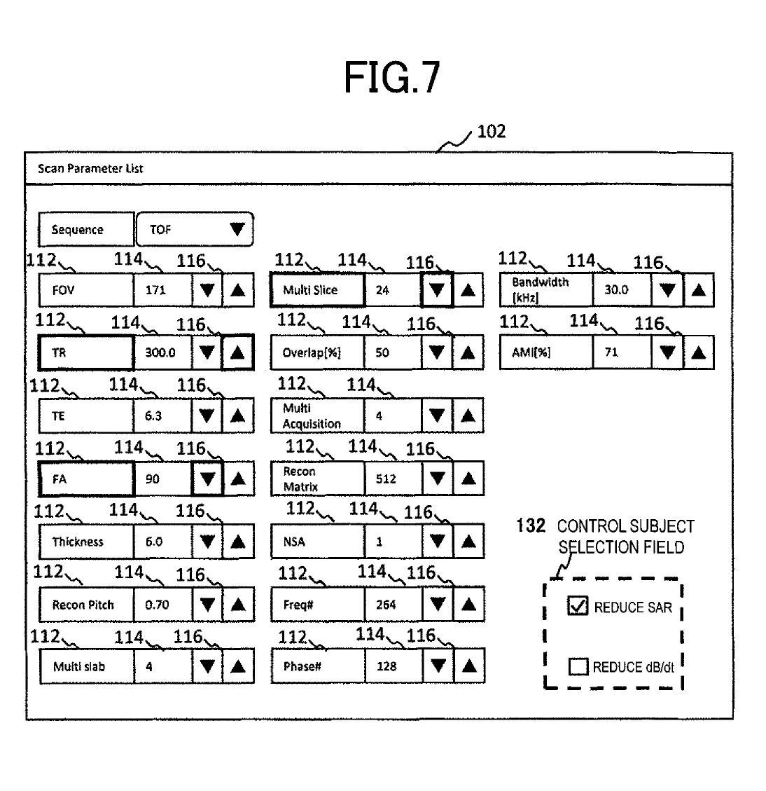

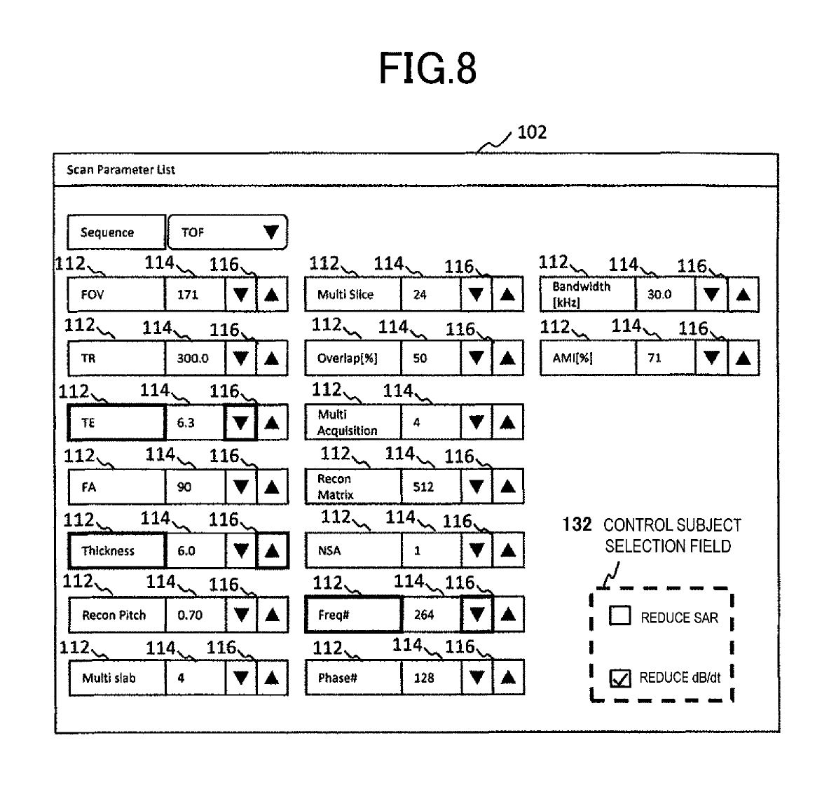

Then, the increase/decrease suggestions are registered in the arithmetic expression data shown in FIG. 5 and are highlighted by emphasizing imaging parameters on the parameter display window with a thick frame as shown FIGS. 7 and 8. The highlighted display is a display suggesting imaging parameters related to calculating control subject values input by a selection operation in the control subject selection field 132, which can distinguish the other displayed imaging parameters by the recognizable suggestion. The highlighted display allows an operator to understand a viewpoint that should be checked precisely and can not only improve the workability but also reduce errors such as misrecognition, which leads to reliability improvement consequently.

Additionally, FIG. 7 is an example of the highlighted display in a case where SAR is selected as a reduction target. Variables “TR”, “Multi Slice”, and “FA” are set as imaging parameters, and increase/decrease suggestions are displayed by the increase button for “TR” and by the decrease button for “Multi Slice” and “FA”.

Also, FIG. 8 is an example of the highlighted display in a case where dB/dt is selected as a reduction target. Variables “TE”, “Freq#”, and “Thickness” are set as imaging parameters, and increase/decrease suggestions are displayed by the decrease button for “TE” and “Freq#” and by the increase button for “Thickness”.

Next, a value change is received for the searched imaging parameters (Step S603). The parameter inquiry section 17 obtains a change value increased/decreased or input by an operator and temporarily stores it in the internal memory 66. Additionally, changing imaging parameters other than highlighted parameters may be prohibited, or a change against increase/decrease suggestions may be prohibited. This can reduce influence of operational errors or misunderstanding by an operator.

When inputting imaging parameters is not finished (Step S604: No), the procedure goes back to Step S602 to update increase/decrease suggestions by setting the changed imaging parameter values as current values and receives the next input.

As described above, according to the first embodiment of the present invention, imaging parameters for reducing either or both of a SAR and a dB/dt are extracted and displayed, and a change plan to increase or decrease the values is presented. Hence, the imaging parameters can be properly changed for the reduction. When an operator inputs at least either one of a SAR or a dB/dt as a reducing item from the control subject selection field 132, the control unit 60 extracts and displays imaging parameters for reducing the input control subject values. The operator can input the displayed imaging parameters as changing targets.

Therefore, appropriate operations can be performed. Additionally, which imaging parameters should be adjusted becomes clear in order to reduce at least either one of a selected SAR or a selected dB/dt. Therefore, the imaging parameter changing method becomes clearly understandable. Additionally, errors are reduced. Particularly in the embodiments shown in FIGS. 7 and 8, selected imaging parameters and increase/decrease directions are highlighted, which can achieve clear understanding and further error reduction.

### Second Embodiment

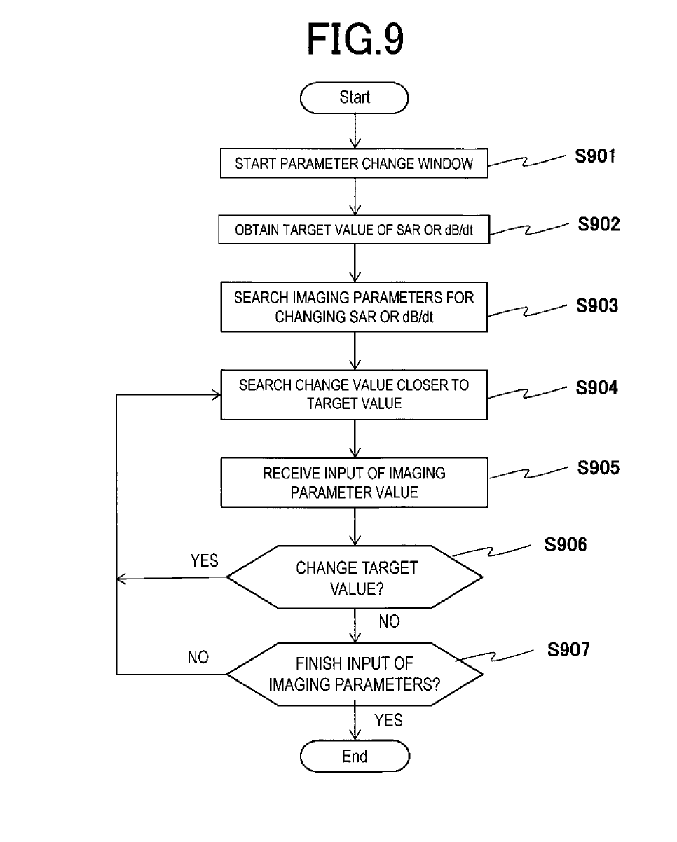

When imaging parameters are changed after setting an allowable value of an imaging index and the like as a target value in advance in a case where an estimated value of a SAR or a dB/dt exceeds the allowable value in the first embodiment, it may be configured so as to avoid exceeding the allowable value by suggestion to approximate to each target value. The other example of Step S304 that realizes the above will be described using FIG. 9.

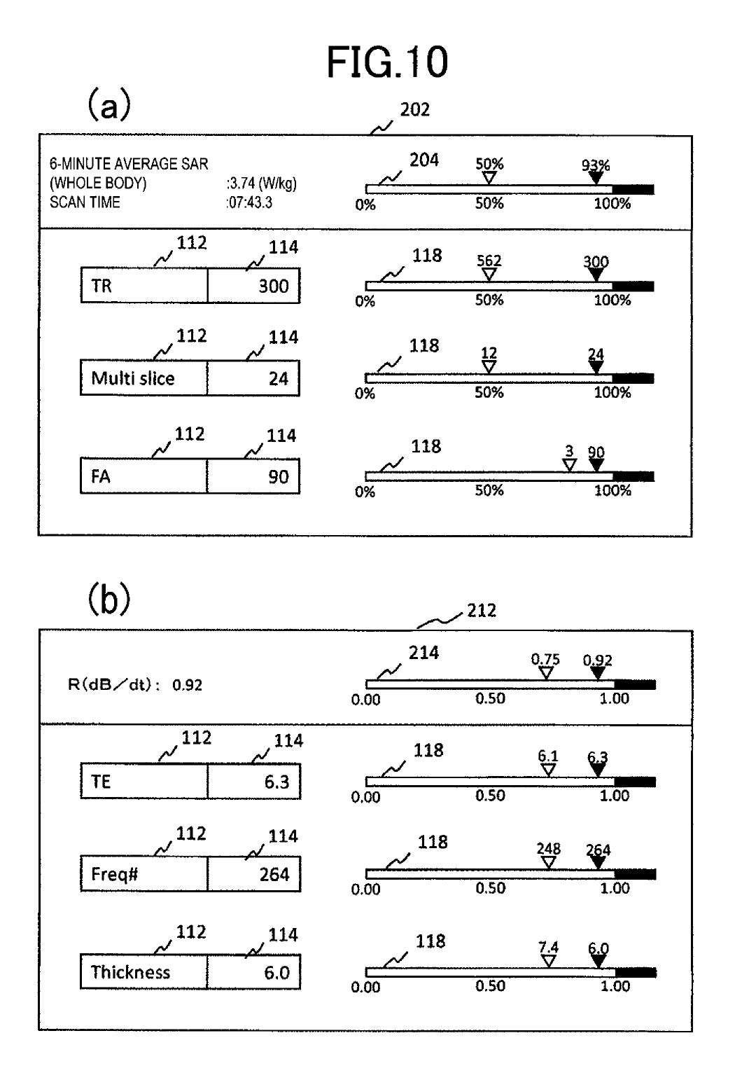

In Step S901, as an image for receiving an imaging parameter change, a parameter change window is started in the present embodiment. When an operator selects a control subject to be reduced in the parameter display window shown in FIG. 4 and the CPU 14 of the control unit 60 identifies the reduction target, the parameter inquiry section 17 separately displays the parameter change window shown in FIG. 10 on the output device 96.

Additionally, FIG. 10(a) is an example of a parameter change window 202 in case of selecting a SAR as a reduction target, and FIG. 10(b) is an example of a parameter change window 212 in case of selecting a dB/dt as a reduction target. The change window 202 for SAR shown in FIG. 10(a) may be displayed separately from the display of FIG. 4 and the displays of FIGS. 7 and 8, may be displayed with the display of FIG. 4 and the displays of FIGS. 7 and 8 on the same screen, or may be displayed using a method of superimposing the display of FIG. 4 and the displays of FIGS. 7 and 8 and displaying a selected display on the front.

In Step S902, a target value of a SAR or a dB/dt is obtained. The parameter inquiry section 17 obtains either target value to be identified as a reduction target. The target value is specified within an allowable value in an operation mode capable of imaging. The setting value may be set for arithmetic expression data shown in FIG. 5 or the like in advance so as to use the setting value or may be input by an operator from the input device 91. For example, in a normal operation mode, the allowable value is set as a target value or the like.

By disposing a progress bar 204 or the like that is one of graphical user interfaces (hereinafter, described as GUI) showing, for example, a relative position on the parameter change window shown in FIG. 10, the obtained target value is displayed with a current value of a SAR or a dB/dt when a reference value (such as 0 to 100%) is set as a standard.

For example, FIG. 10(a) shows that a SAR current value is 93% (the position specified by the black triangle) and that the target value is 50% (the position specified by the white triangle) in a case where a 6-minute average SAR (whole body) is 3.74 (W/kg) when an allowable value in a first level controlled operating mode is set to 100% and an allowable value in a normal operation mode is set to 50% as the reference values. That is, in this embodiment, a percentage to the maximum value of the reference value is displayed, and additionally, the percentage is displayed in a linear graph.

The current value is 93% to the maximum value of the reference value and is displayed with the black triangle mark as an example as shown in the diagram. Also, the target value is displayed with the white triangle mark as shown in the diagram. By describing the current value and the target value with the different marks, a current state can be judged properly, which can reduce misjudgment. The above is also similar to the parameter change window 212 for a dB/dt shown in FIG. 10(b).

A current value and a target value are described using a GUI, for example, a progress bar 214. The current value and the target value are displayed in percentage to the maximum value of the reference value. The current value is displayed with the black triangle mark as an example as shown in the diagram. Also, the target value is displayed with the white triangle mark as shown in the diagram. By describing the current value and the target value with the different marks, a current state can be judged properly, which can reduce misjudgment.

Imaging parameters related to changing a SAR or a dB/dt that is a control subject are searched (Step S903) using the database 150 described in FIG. 5. The parameter inquiry section 17 identifies imaging parameters similarly to Step S601, and the imaging parameters and currently set parameter values are displayed on the parameter change windows 202 and 212 as shown in FIG. 10.

Next, imaging parameters value are searched so that a current SAR or dB/dt value approximates more to a target value (Step S904). The parameter inquiry section 17 calculates suggestion values of the imaging parameters that should be changed to obtain a target SAR or dB/dt value by increasing or decreasing the respective imaging parameter values. The suggestion values (suggested by the white triangle) are displayed with current values (suggested by the black triangle) of the imaging parameters similarly to the target value by disposing progress bars or the like on the parameter change windows 202 and 212 as shown in FIG. 10. In this embodiment, as an example, values are displayed in percentage, and target values are also presented.

In an example shown in FIG. 10(a), it is understood that an imaging parameter “TR” should be increased from the current value 300 to the suggestion value 562, that an imaging parameter “Multi Slice” should be decreased from the current value 24 to the suggestion value 12, and that an imaging parameter “FA” should be decreased from the current value 90 to the suggestion value 3 in order to decrease a SAR from the current value 93% to the target value 50%.

Also, in an example shown in FIG. 10(b), it is understood that an imaging parameter “TE” should be decreased from the current value 6.3 to the suggestion value 6.1, that an imaging parameter “Freq#” should be decreased from the current value 264 to the suggestion value 248, and that an imaging parameter “Thickness” should be increased from the current value 6.0 to the suggestion value 7.4 in order to decrease a dB/dt from the current value 0.92 to the target value 0.75.

Next, inputting parameter values is received for the searched imaging parameters (Step S905). The parameter inquiry section 17 obtains change values based on positions specified using progress bars or the like by an operator and temporarily stores the value in the internal memory 66. When the operator inputs imaging parameter values, new values may be input in the setting value fields 114 provided corresponding to the item fields 112 of the imaging parameters, or positions corresponding to values to be input may be specified using progress bars 118.

By specifying positions corresponding to values to be input on the progress bars 118 using a touch panel or a pointing device, the control unit 60 calculates the values corresponding to the specified positions and displays the calculation results in the setting value fields 114. When an operator specifies the positions on the progress bars 118, numerical values corresponding to the specified positions are displayed in the setting value fields 114, and the operator moves the specified positions along the progress bars 118 and stops at desired positions so as to display desired values while checking the numerical values displayed in the setting value fields 114. Thus, the desired values can be input as imaging parameter values.

When a position shown by the white triangle is set as a specified position, the position shown by the white triangle should be selected. This method is superior in understanding the current status because imaging parameter values can be specified based on relationships with current values and target values. An operator may input change values for all the imaging parameters related to a SAR or a dB/dt to be specified for which imaging parameters are now being changed. However, in case of showing a SAR to be specified as an example, it may be configured so that the control unit 60 calculate a value of the imaging parameter “FA” based on the fact that values of the imaging parameters “TR” and “Multi slice” have been set.

In case of changing target values (Step S906: Yes) or in case of continuing input of imaging parameters (Step S907: No), change values approximating target values more are searched by going back to Step S904, and then the next input is received.

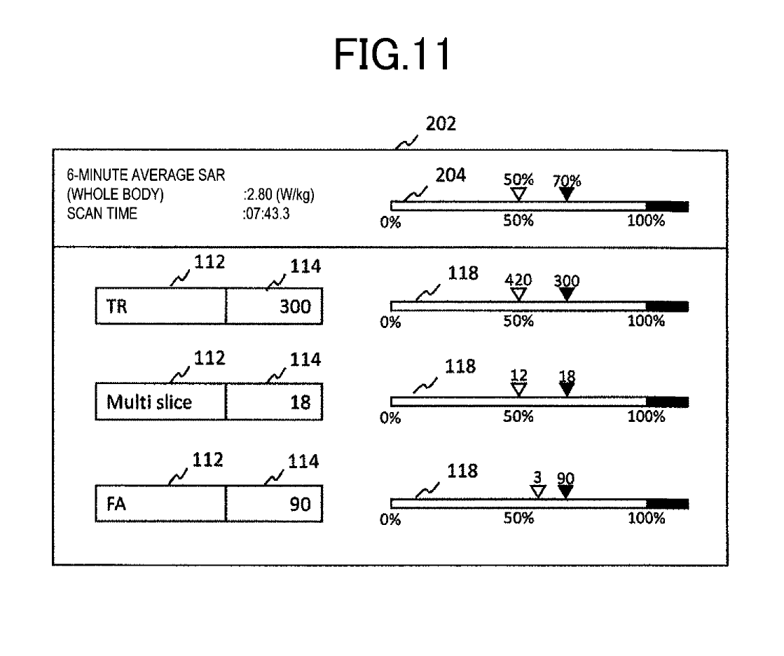

That is, when the parameter inquiry section 17 obtains change values of imaging parameters (Step S907: No), the calculation control section 18 of the CPU 14 included in the control unit 60 calculates SAR and dB/dt values using the change values temporarily stored in the internal memory 66, further re-searches imaging parameter values at which a SAR or a dB/dt most approximates a target value for each imaging parameter by going back to Step S904, and then updates a parameter change window as shown in FIG. 11.

For example, when “Multi slice” is changed from the value 24 to the value 18, the SAR is decreased from 93% to 70%, and, in addition to this, the suggestion value of “TR” for approximating the SAR to 50% that is the target value is updated to 420. Additionally, a relative position can be changed for a parameter whose value is not changed.

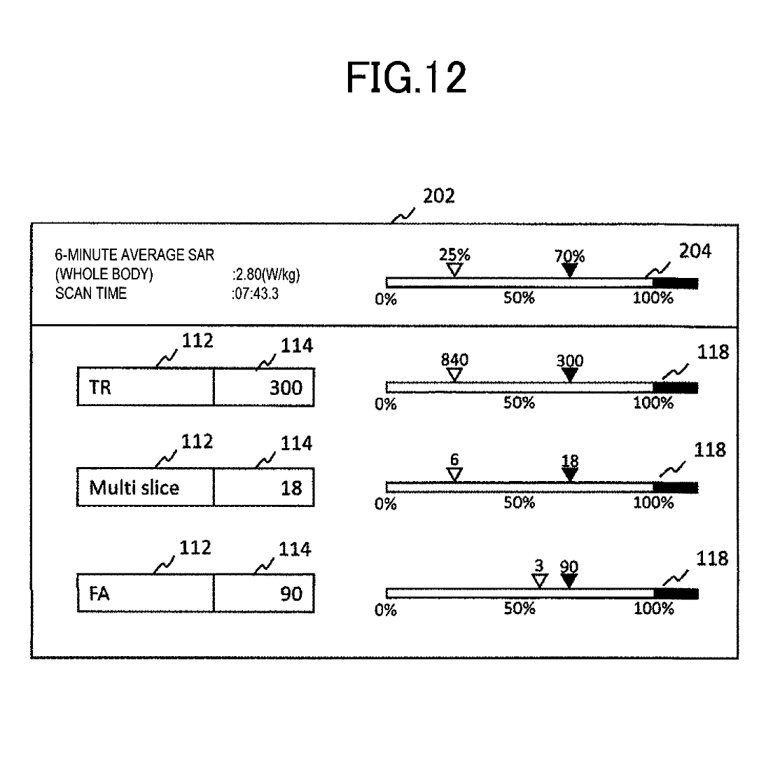

Also, the calculation control section 18 of the CPU 14 calculates SAR and dB/dt values when target values are changed (Step S906: Yes), further re-searches imaging parameter values at which a SAR or a dB/dt more approximates a target value for each imaging parameter by going back to Step S904, and then updates a parameter change window as shown in FIG. 12.

For example, in case of decreasing a SAR target value to 25%, the suggestion value of “TR” for approximating the SAR to the target value is updated to 840, and then the suggestion value of “Multi Slice” is updated to 6.

As described above, according to the second embodiment of the present invention, recommended parameter values for achieving a SAR or dB/dt target value can be changed with reference to them. This makes a parameter adjustment method for reducing a SAR and a dB/dt understandable, and imaging parameters can be adjusted efficiently so that a SAR and a dB/dt in a pulse sequence enters a state in an operation mode range capable of imaging.

The embodiments described in FIGS. 10(a), 10(b), 11, and 12 are contrast to allowable values in a first level controlled operating mode to be specified, current values and target values to be specified are displayed. Particularly, an allowable value in the first level controlled operating mode to be specified is set as 100% and is displayed in a bar graph. Although a general object is imaged in a first level controlled operating mode, an object requiring special care is imaged with a smaller burden by setting the first level controlled operating mode as a standard.

In this case, by setting target values at a percentage to allowable values in the first level controlled operating mode to be specified, more appropriate imaging parameters can be set, which can also obtain higher reliability in imaging. In the present embodiment, when target values are set at a percentage to allowable values in the first level controlled operating mode to be specified, the control unit 60 sets and displays setting values of the related imaging parameters from the target values.

Using such a method, appropriate imaging parameter values can be set. Some improve a control subject by increasing setting values of imaging parameters, and others improve a control subject by decreasing the setting values of imaging parameters. In the present embodiment, certain criteria are set in an improvement direction of an axis of the progress bar 204 showing a current value and a target value to be specified and in an arrangement direction of axes of the bar progress bars 118 showing current values and target values of the related imaging parameters.

As an example, all the left sides of the progress bars 204 and 118 shown in FIGS. 10(a), 10(b), 11 and 12 shows the improvement directions of control subjects. Thus, the progress bars 118 are not set based on the magnitudes of numerical values of the axes, but the axis directions of the progress bars 204 and 118 are set based on the improvement directions of control subjects, which effectively enables easy operation.

### Third Embodiment

Although reference values of the progress bars 118 or the like are displayed in percentage (relative display such as an allowable value in the first level controlled operating mode: 100% and allowable value in the normal operation mode: 50%) on the parameter change windows 202 and 212 to be used for changing imaging parameters in the second embodiment, the reference values may be displayed in the other method.

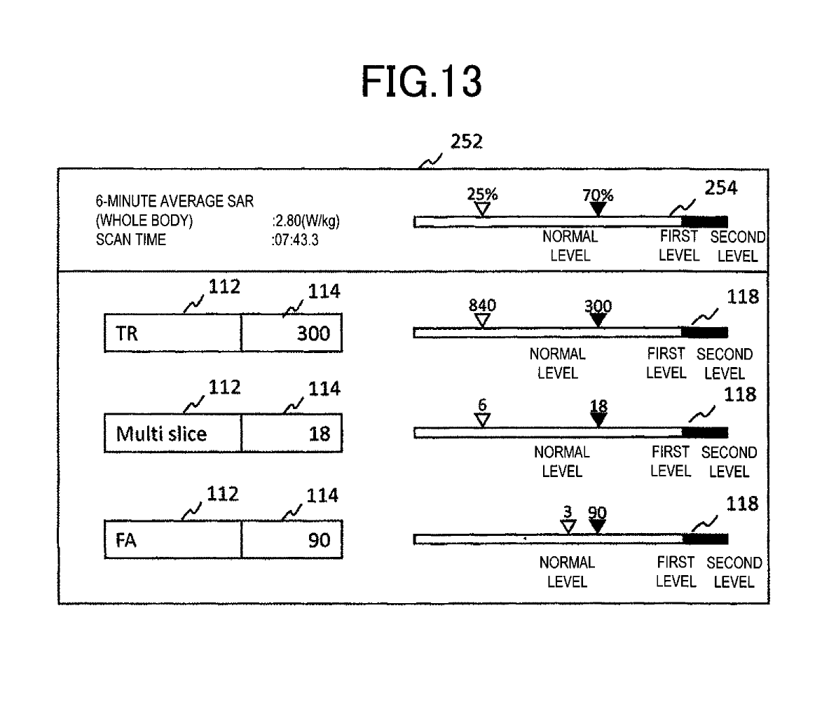

For example, as shown in FIG. 13, level suggestions such as “NORMAL LEVEL” in a position corresponding to an allowable value in the normal operation mode, “FIRST LEVEL” in a position corresponding to an allowable value in the first level controlled operating mode, and “SECOND LEVEL” in a position corresponding to an allowable value in the second level controlled operating mode may be displayed.

As described above, according to the third embodiment of the present invention, it becomes easier to understand whether or not a SAR or a dB/dt to be specified enters a state in a range capable of imaging in imaging based on a pulse sequence, which can adjust imaging parameters effectively. For example, a level of a SAR or dB/dt to be specified is set for imaging, and burdens are considered for a state of the object 11, which makes determination easier and is also superior in management.

As an example, it is difficult to show a state of the object 11 with detailed numbers, and the effect is small even if the state is shown in percentage in detail. When the management is performed with three-stage classification or the like, desired results can be obtained.

### Fourth Embodiment

The second and third embodiments described a method in which an operator changes imaging parameters by displaying the parameter change window 202 or 212 separately from the parameter display window 102. Although the parameter display window 102 and a parameter change window may be displayed separately, it may be configured so that an operator changes the imaging parameters by displaying the parameter display window 102 and the parameter change windows 202 and 204 of a SAR and a dB/dt on the same display at the same time.

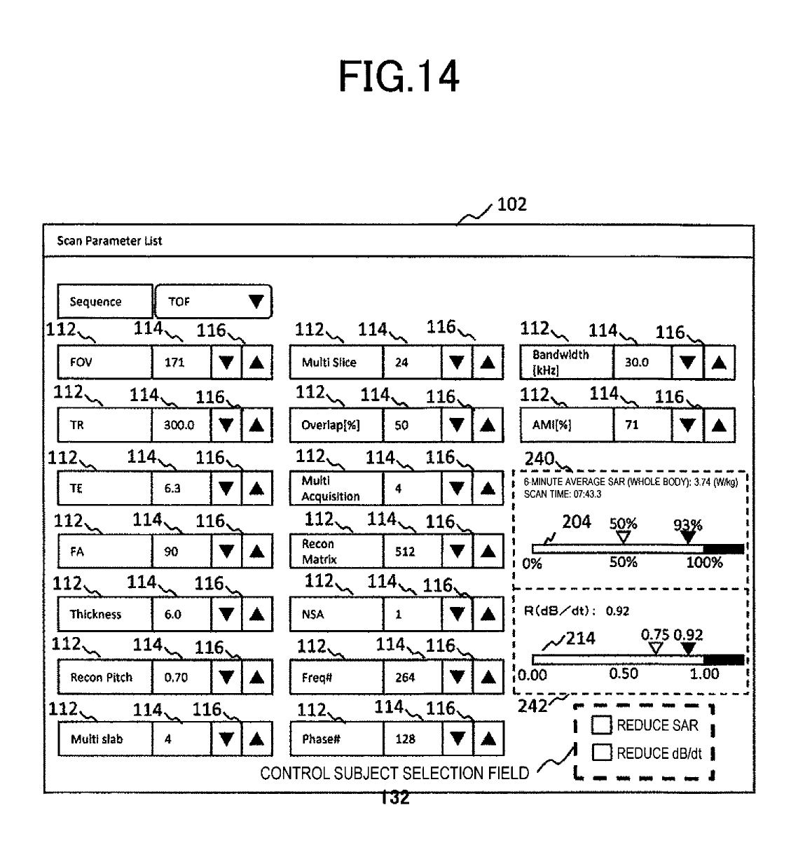

For example, as shown in FIG. 14, progress bars displayed on a parameter change window or the like are arranged in a free region other than the imaging parameter display regions of a parameter display window, and current values and target values are displayed relatively to reference values (such as 0 to 100%). FIG. 14 shows the free region with broken-line frames 240 and 242.

Additionally, although regions displaying the progress bar 204 for a SAR and the progress bar 214 for a dB/dt may be respectively provided by providing the broken-line frames 240 and 242 respectively, it may be configured so that the progress bar 204 for a SAR and the progress bar 214 for a dB/dt are displayed by sharing one region. In this case, a control subject selected in the control subject selection field 132, i.e. the progress bar 204 or 214 selected in the control subject selection field 132 will be displayed.

Current and target values may be highlighted in case of selecting reduction targets. That is, it may be configured so that current and target values of a SAR are displayed in case of selecting a SAR as a reduction target and so that current and target values of a dB/dt are displayed in case of selecting a dB/dt as a reduction target based on selection in the control subject selection field 132.

As described above, according to the fourth embodiment of the present invention, it becomes easier to understand whether or not a SAR and a dB/dt in a pulse sequence enter a state in an operation mode range capable of imaging only with a parameter display window, which can adjust imaging parameters effectively.

Also, a parameter display region displaying imaging parameters, a target value display region of control subjects shown in the broken-line frames 240 and 242, and a control subject selecting region for displaying the control subject selection field 132 are provided. At least imaging parameters related to a SAR to be specified, the current values, and the operation fields 116 that suggest the change directions and allow an input operation as needed are arranged and displayed in the parameter display window.

In the target value display region of control subjects, for example, target values of the control subjects to allowable values are displayed in percentage as shown with the progress bars 204 and 214. Although the progress bar 204 for a SAR and the progress bar 214 for a dB/dt may be displayed comparatively as described above, a progress bar selected in the control subject selection field 132 may be displayed selectively. When the progress bar 204 for a SAR and the progress bar 214 for a dB/dt are displayed comparatively, this is effective to easily understand the entire state. On the other hand, when the progress bar 204 for a SAR and the progress bar 214 for a dB/dt are displayed selectively, this has an advantage in that a region required for display is small.

### Fifth Embodiment

Although imaging parameters are set for arithmetic expression data or the like shown in FIG. 5 and extracted as imaging parameters to be used for SAR and dB/dt calculations in the first to fourth embodiments, it may be configured so that an operator can arbitrarily select imaging parameters to be changed.

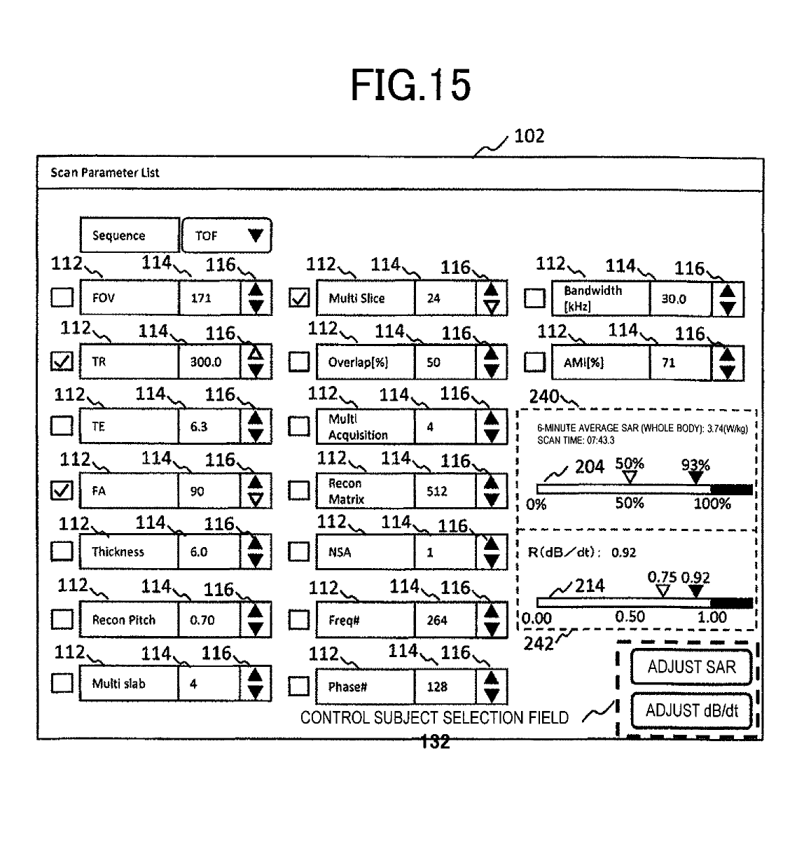

As shown in FIG. 15, check boxes (one of GUIs to be used for selecting a plurality of items) are provided for each imaging parameter in a parameter display window, and an operator specifies whether or not each imaging parameter is set as a change target using the input device 91. Additionally, when a parameter change is performed in the parameter display window, up and down buttons are enabled only for checked imaging parameters, and it should be set so that the values can be increased and decreased. Also, when a SAR or dB/dt button shown in the lower right of the diagram is pressed down, it should be set so as to display and change only the imaging parameters checked in a parameter change window. That is, the imaging parameters selected by an imaging parameter selection operation such as checking as described above becomes targets whose setting values can be changed.

Also, the operation fields 116 suggesting an increase/decrease direction are displayed for the selected imaging parameters. For example, an increase is suggested by an upward triangle for the selected imaging parameter “TR”. A decrease is suggested by a downward triangle for the selected imaging parameters “FA” and “Multi slice”.

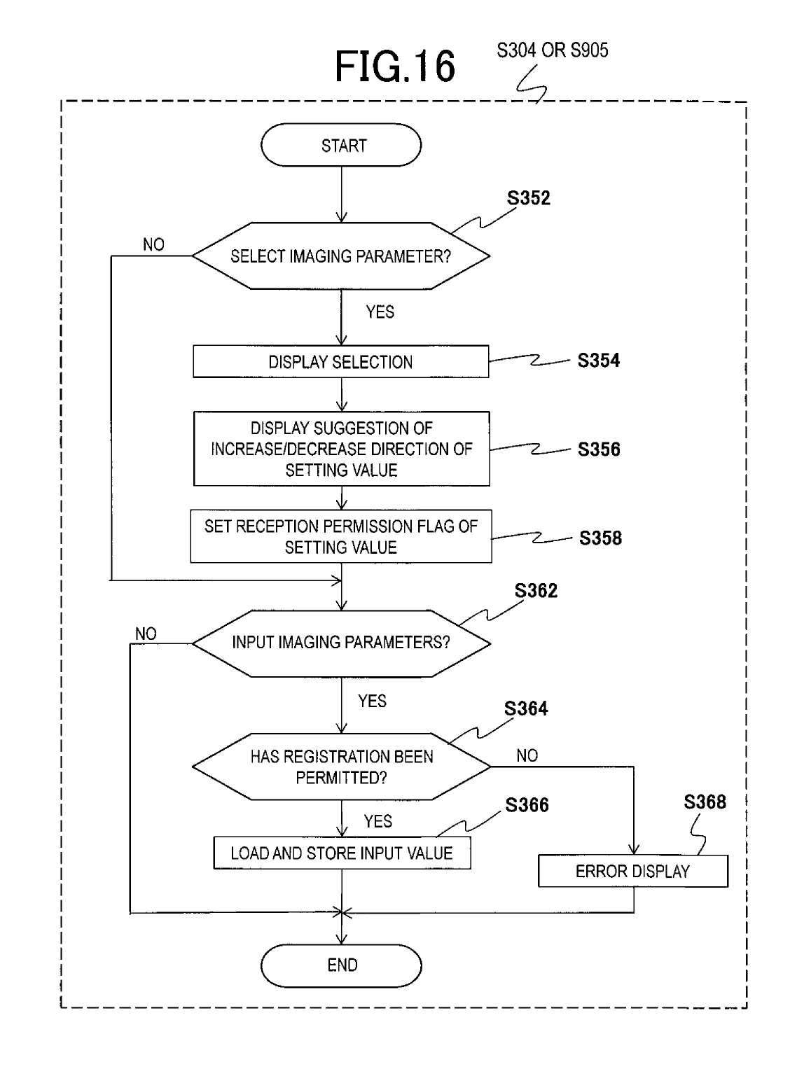

FIG. 16 shows a flow chart for executing the operation described in FIG. 15, and the flow chart is executed by the control unit 60. In the flow chart shown in FIG. 16, a process corresponding to Step S304 described in FIG. 3 and STEP S905 described in FIG. 9 is performed. When an operator performs an operation of selecting imaging parameters shown in FIG. 15, this flow chart is executed by setting the operation as a start condition, and when the operator performs an operation of inputting an imaging parameter value shown in FIG. 15, this flow chart is further executed by setting the operation as a start condition.

Alternatively, as the other starting method, the process may be performed based on whether or not an operator selects imaging parameters shown in FIG. 15 or whether or not the operator performs an input operation for imaging parameter values between ending the last flow chart execution and starting the next flow chart execution by repeatedly starting this flow chart at a certain time interval.

When the flow chart execution shown in FIG. 16 starts using the above method, whether or not it is the imaging parameter selection described in FIG. 15 is determined in Step S352. For example, when it is not the imaging parameter selection but the imaging parameter value input, Steps from S354 to S358 are not required, and the control unit 60 proceeds to Step S362. On the other hand, in case of the imaging parameter selection, the procedure proceeds to Step S354, and a display suggesting being selected is performed. For example, when the imaging parameter “TR” is selected, a check suggesting being selected is displayed.

Also, Step S356 is executed in order to suggest change directions of selected imaging parameter values. For example, in case of the imaging parameter “TR”, the upward triangle showing the increase direction displayed in the operation field 116 is changed to a white triangle. Additionally, in order to permit an input operation of numerical values of selected imaging parameters, Step S358 is executed, and then a reception permission flag of setting values is set. The reception permission flag shows whether or not the numerical value input of the imaging parameter “TR” is permitted.

Next, the procedure proceeds to Step S362. Additionally, although this flow chart describes Step 352 to be executed following Step S358, a target to be processed is different between selecting imaging parameters to change setting values and inputting the selected imaging parameter values, and the selection and input operations may be performed separately under different execution conditions.

In this case, the procedure stops in Step S358, and Step S362 may be set so as to start under new starting conditions. When Step S362 is executed by the control unit 60, whether or not to input numerical values that are imaging parameter setting values is determined. In case of inputting the numerical values of the imaging parameters, whether or not to permit reception of the numerical values is determined in Step S364. When a flag is set in Step S358 described above, it is determined that receiving the numerical values has been permitted, and the numerical values input in Step S366 are loaded and stored in a predetermined memory address.

The stored numerical values are used for calculating a SAR or a dB/dt in the calculation control section 18 described in FIG. 3 for example. On the other hand, Step S362 is different from inputting imaging parameter numerical values, this flow chart ends. Also, when a numerical value change is not permitted in Step S364 even in case of inputting numerical values for imaging parameters, an error is displayed in Step S368, and then this flow chart ends. Even when this flow chart ends, it is repeatedly executed when the starting conditions are satisfied.

As described above, because changeable imaging parameters are not fixed according to the fifth embodiment, an operator can arbitrarily set imaging parameters to be changed in case of narrowing the imaging parameters, which makes the parameter adjustment method for reducing a SAR and a dB/dt understandable.

### Sixth Embodiment

In the fifth embodiment, an operator arbitrarily select imaging parameters to be changed and changes them on a parameter display window. In this case, some changing patterns are prepared in accordance with a plan, a changing pattern is selected in accordance with the plan, imaging parameters intercorrelated with each other are automatically selected based on the selected pattern, and then imaging parameters may be automatically determined in accordance with the above plan.

There are change plans, for example, that contrast is not changed so that the contrast of an image to be obtained becomes as stable as possible, that a scan time is not changed and, in particular, is not extended, and that the number of images to be obtained is not changed, that is, the number of images to be imaged is maintained, and the like. According to such plans, groups of imaging parameters based on the respective plans are set as the change plans.

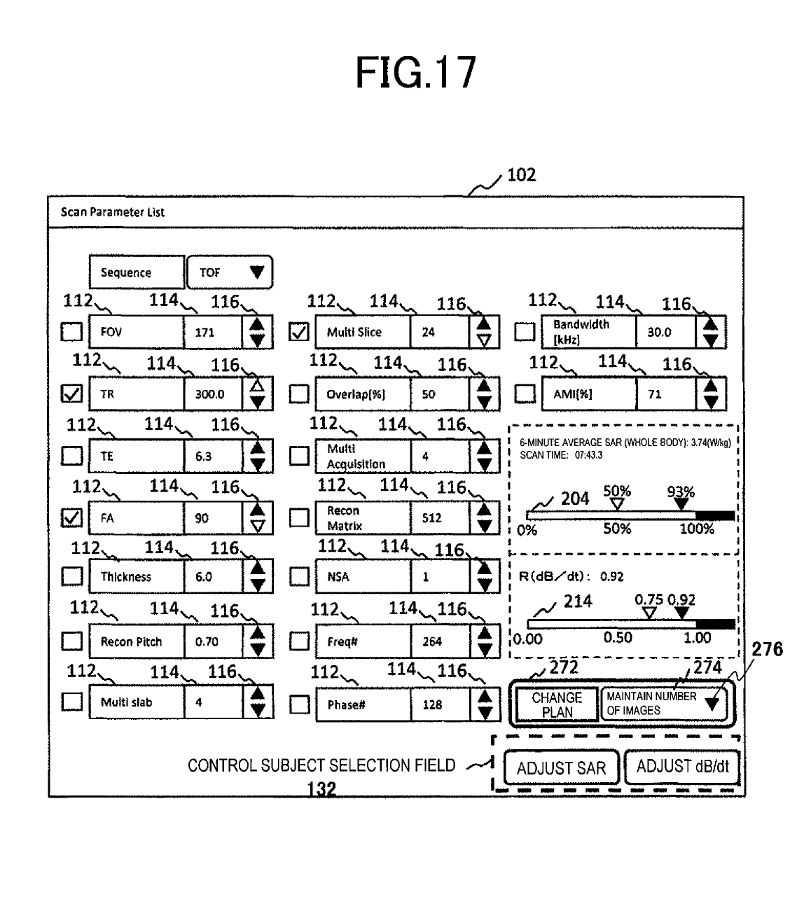

As shown in FIG. 17, a change plan display region 272 displaying a change plan is provided on the parameter display window 102, and a change plan list 274 listing change plans is disposed on the change plan display region 272. An operator selects an appropriate change plan from the change plan list 274. A database in which list contents are described in the external storage device 61 in advance is created for a change plan list, and change plans composing the above list are displayed in order by operating a pull-down display 276 in the change plan display region 272 for example. By selecting a change plan thus, imaging parameters are automatically selected in accordance with the change plan. For example, a check showing that the imaging parameter “TR” is selected is displayed. Off course, the color may be changed instead of displaying a check.

Additionally, similarly to the operation of the pull-down display 276 and the like, it may be configured so that an operator can select an appropriate change plan from the change plan list 274 by a change plan selection operation and further increase and decrease imaging parameters to be changed arbitrarily. For example, if a group of imaging parameters is “TR” and “FA” when a change plan is specified in a case where only “TR” should be changed, “FA” may be excluded from the group by an excluding operation.

As described above, according to the sixth embodiment of the present invention, a SAR and a dB/dt can be reduced by adjusting parameters in light of a contrast, an imaging time, and a resolution of an image to be obtained. That is, an operator can easily adjust parameters to reduce a SAR or a dB/dt under desired conditions.

### Seventh Embodiment

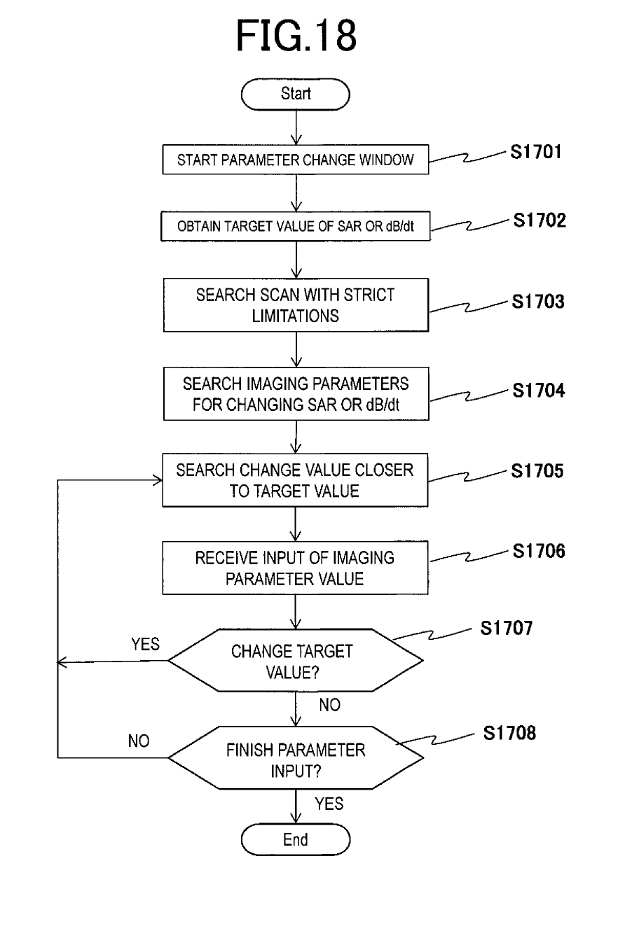

Although an operator selects a desired pulse sequence from a plurality of pulse sequences in a protocol in the first to sixth embodiments, a specific example of Step 4 in case of selecting a plurality of pulse sequences to be changed will be described using FIG. 18. For example, a case where two scans of the imaging sites “Pelvis” and “Knee” are set as targets to be changed and the like are included.

A parameter change window is started to receive an imaging parameter change (Step S1701). When the CPU 14 identifies a reduction target selected by an operator in a parameter display window, the parameter inquiry section 17 displays the parameter change window shown in FIG. 18 on the display 98 of the output device 96.

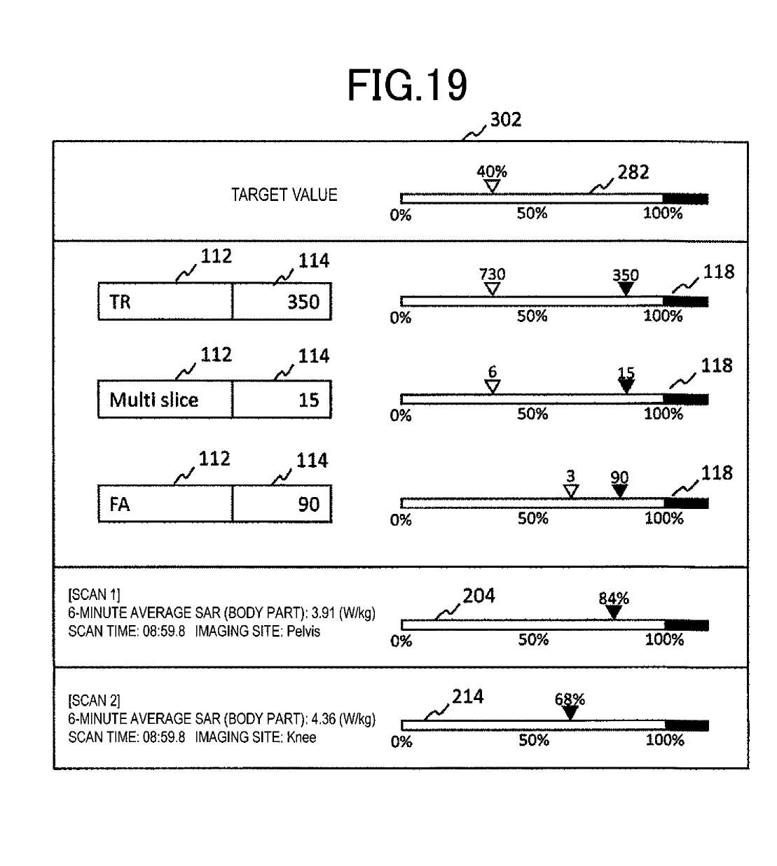

A target value of a SAR or a dB/dt is obtained (Step S1702). The obtained target value is displayed using a progress bar 282 or the like in a parameter change window 302 shown in FIG. 19. The reference value 40% is displayed as a standard in the position shown by the white triangle. Current values of a SAR or a dB/dt are displayed using the progress bars 204 and 214 or the like similarly in the positions, for example, shown by the black triangles for each scan (pulse sequence).

The most severely limited scan is searched (Step S1703). The calculation control section 18 calculates a SAR and dB/dt values for all the pulse sequences selected as change targets, further calculates percentages to allowable values, and then stores them in the internal memory 66. Then, the CPU 14 identifies a calculated highest percentage from among them as the most severely limited scan.

Imaging parameters reducing a SAR or a dB/dt are searched for the most strictly limited scan (Step S1704) in order to search imaging parameter values at which the SAR or the dB/dt becomes the closest to a target value (Step S1705).

The parameter inquiry section 17 displays imaging parameters and the parameter values (current values) on a parameter change display as shown in FIG. 19 and displays suggestion values (positions shown by the white triangles) and current values (positions shown by the black triangles) of imaging parameters that should be changed to obtain target values of a SAR or a dB/dt on the progress bars 118 or the like.

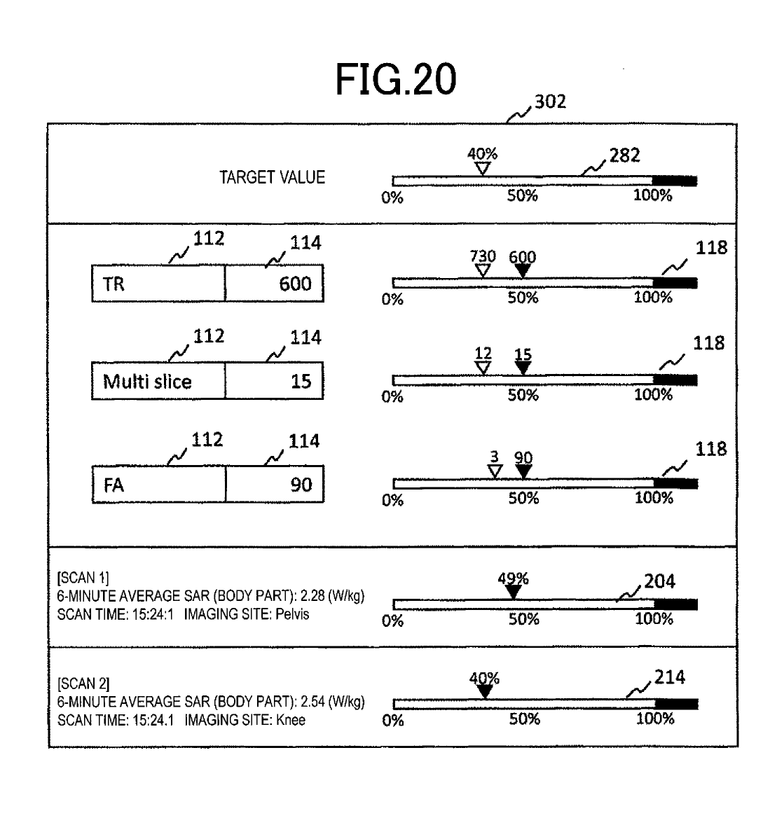

Next, parameter value input is received (Step S1706). In case of changing target values (Step S1707: Yes) or in case of not finishing imaging parameter input (Step S1708: No), the procedure goes back to Step S904 to search change values that are the closest to target values, and a parameter change display is updated to receive the next input as shown in FIG. 20. FIG. 20 is a window showing a state after the imaging parameters are changed, and the imaging parameter “TR” value is changed from 350 to 600.

As described above, according to the seventh embodiment, a SAR and a dB/dt of not only the whole body but also each body part are considered, which can adjust efficiently adjust imaging parameters so that a SAR and a dB/dt of a pulse sequence are a state within an operation mode range capable of imaging.

Also, parameter adjustment to reduce a SAR or a dB/dt can be performed without changing a contrast between each scan.

The present invention is not limited to the above described embodiments.

### REFERENCE SIGNS LIST

11: object
12: sequencer
14: CPU
15: display processing section
16: operation reception section
17: parameter inquiry section
18: calculation control section
20: static magnetic field generation source
30: gradient magnetic field generating unit
32: gradient magnetic field coil
34: gradient magnetic field power source
40: high-frequency irradiation unit
42: high-frequency oscillator
44: modulator
46: high-frequency amplifier
48: irradiation coil
50: signal detection unit
52: reception coil
54: signal amplifier
56: quadrature phase detector
58: A/D converter
60: control unit
61: external storage device
62: optical disk
64: magnetic disk
66: internal memory
67: protocol data
68: parameter data
69: arithmetic expression data
70: SAR measurement section
80: bed moving device
82: bed
90: input/output device
91: input device
92: pointing device
94: keyboard
96: output device
98: display
99: printer
100: MRI apparatus

1. A magnetic resonance imaging apparatus comprising:
a static magnetic field generation source that generates a static magnetic field in a space accommodating an object;
a gradient magnetic field generating unit that generates a gradient magnetic field to be superimposed on the static magnetic field;
a high-frequency magnetic field generating unit for irradiating a high-frequency magnetic field pulse to the object;
a signal detection unit that detects a nuclear magnetic resonance signal to be generated from the object;
a sequencer that controls the static magnetic field generation source, the gradient magnetic field generating unit, the high-frequency magnetic field generating unit, and the signal detection unit according to a pulse sequence; and
a control unit that has a storage device, an input device, an output device, and a CPU,
wherein, when a control subject is input based on an input operation, the control unit displays suggestions of imaging parameters related to the control subject and change directions of the imaging parameters, and
wherein, when the displayed imaging parameters are changed, the control unit further calculates values of the selected control subjects based on the changed imaging parameter values.

2. The magnetic resonance imaging apparatus according to claim 1,
wherein, when the control subject input based on the input operation is a specific absorption rate, the control unit displays a suggestion of imaging parameters related to the specific absorption rate on the output device and further displays information about a suggestion of the change directions of the imaging parameters on the output device.

3. The magnetic resonance imaging apparatus according to claim 1,
wherein, when the control subject input based on the input operation is a magnetic field variation rate per unit time of a magnetic flux density, the control unit displays a suggestion of imaging parameters related to the magnetic field variation rate per the unit time on the output device and further displays information about a suggestion of change directions of the imaging parameters for reducing the magnetic field variation rate on the output device.

4. The magnetic resonance imaging apparatus according to claim 1,
wherein imaging parameters including those related to a specific absorption rate and a magnetic field variation rate per unit time of a magnetic flux density are displayed on the output device,
wherein, when the control subject input based on the input operation is a specific absorption rate, the control unit highlights the imaging parameters related to the specific absorption rate from among the imaging parameters displayed on the output device to distinguish from the other imaging parameters, and
wherein when the control subject input based on the input operation is a magnetic field variation rate per unit time of a magnetic flux density, the control unit highlights the imaging parameters related to the magnetic field variation rate per the unit time from among the imaging parameters displayed on the output device to distinguish from the other imaging parameters.

5. The magnetic resonance imaging apparatus according to claim 1,
wherein at least one of a specific absorption rate or a magnetic field variation rate per unit time is displayed as a control subject on the output device, and a plurality of imaging parameters related to the control subject are further displayed on a display surface of the output device,
wherein a current value of the control subject and each current value of the plurality of imaging parameters are displayed respectively in accordance with a display of the control subject and each of the plurality of imaging parameters, and
wherein, when a target value of the control subject is set, each value of the plurality of imaging parameters is displayed in accordance with the set target value.

6. The magnetic resonance imaging apparatus according to claim 5,
wherein a current value and a target value of the control subject that is composed of at least one of the specific absorption rate or the magnetic field variation rate per unit time are displayed relatively to allowable values.

7. The magnetic resonance imaging apparatus according to claim 6,
wherein the plurality of imaging parameters related to the control subject includes a first imaging parameter whose value increase results in improvement of the control subject and a second imaging parameter whose value decrease results in improvement of the control subject,
wherein current values of the control subject, the first imaging parameter, and the second imaging parameter are arranged and displayed respectively with progress bars, and
wherein, according to an improvement direction of an axis of the progress bar for the current value of the control subject, the increase direction of the axis of the progress bar showing the current value of the first imaging parameter is disposed, and the decrease direction of the axis of the progress bar showing the current value of the second imaging parameter is further disposed.

8. The magnetic resonance imaging apparatus according to claim 6,
wherein a current value and a target value of the control subject are displayed using a plurality of levels set for the allowable values.

9. The magnetic resonance imaging apparatus according to claim 1,
wherein an imaging parameter display region displaying a plurality of imaging parameters and a target value display region displaying at least one of target values of a specific absorption rate or a magnetic field variation rate per unit time of a magnetic flux density are provided on the display window of the output device,
wherein imaging parameters related to a specific absorption rate and imaging parameters including imaging parameters related to a magnetic field variation rate per unit time of a magnetic flux density are arranged and displayed respectively on the imaging parameter display region, and
wherein at least one of target values of a specific absorption rate or a magnetic field variation rate per unit time of a magnetic flux density is displayed in a relative relationship with the allowable value on the target value display region.

10. The magnetic resonance imaging apparatus according to claim 9,
wherein a control subject selecting region that displays a control subject selection field for selecting a specific absorption rate or a magnetic field variation rate per unit time of a magnetic flux density is provided on the display window of the output device, and a target value of a control subject selected based on the control subject selection field displayed in the control subject selecting region is displayed in a relative relationship with the allowable value on the target value display region.

11. The magnetic resonance imaging apparatus according to claim 1,
wherein a selected state is displayed by the control unit based on selection operations for a plurality of the imaging parameters related to the specific absorption rate displayed on the output device, and then the setting value input is permitted.

12. The magnetic resonance imaging apparatus according to claim 1,
wherein a change plan list is displayed in a change plan display region displaying change plans of imaging parameters, and then the relationships to imaging parameters related to a selected change plan are displayed based on selecting the change plan from the list.

13. The magnetic resonance imaging apparatus according to claim 1,
wherein, when a plurality of the pulse sequences are specified, a control unit calculates a control subject value in each of the pulse sequences and sets a pulse sequence whose control subject value is in the most severe state as a change target.

14. The magnetic resonance imaging apparatus according to claim 13,
wherein control subject values in the plurality of pulse sequences are displayed on a display window, and additionally, imaging parameter values related to control subjects are displayed.

15. An imaging parameter setting assisting method of a magnetic resonance imaging apparatus,
wherein a static magnetic field and a gradient magnetic field are generated in a space accommodating an object according to control of a sequencer operating based on a pulse sequence, a high-frequency magnetic field pulse is irradiated to the object, and then a nuclear magnetic resonance signal to be generated from the object is detected,
wherein a control unit that has a storage device, an input device, an output device, and based on an input control subject, a CPU displays suggestions of imaging parameters related to the control subject and change directions of the imaging parameters, and
wherein, when the displayed imaging parameter values are further changed, values of the selected control subjects are calculated based on the changed imaging parameter values.
